(19) United States
(12) Patent Application Publication
(10) Pub. No.: US 2013/0295052 A1
(43) Pub. Date: Nov. 7, 2013

(54) NOVEL CONJUGATES FOR TARGETED DRUG DELIVERY

(71) Applicant: Manu Chaudhary, Panchkula (IN)

(72) Inventor: Manu Chaudhary, Panchkula (IN)

(21) Appl. No.: 13/988,428

(22) PCT Filed: Nov. 21, 2011
(86) PCT No.: PCT/IN11/00807

§ 371 Date: Jul. 8, 2013

(30) Foreign Application Priority Data
Nov. 19, 2010 (IN) 2760/DEL/2010

(51) Int. Cl. A61K 47/48

(52) U.S. Cl. CPC A61K 47/48269
USPC 424/85.7; 530/351

(57) ABSTRACT

The present invention relates to a novel drug delivery system comprising a drug(s)-protein-polymer triple conjugate. The triple conjugate employs a (i) protein moiety capable of binding selectively to a particular target site possessed by a cell/affected organ, (ii) a polymer moiety, covalently linked to the protein and (iii) an active drug moiety that includes one or more drug(s) covalently linked to either said polymer moiety or to a protein moiety. The conjugates of the present invention have target specificity and better selectivity to a defined population of cells/organs(s). The present invention further relates to methods of preparation and methods of treatment comprising administering said conjugate as a single unit. The conjugates of the present invention are usefully employed in therapeutic as well as non-therapeutic, e.g., diagnostic applications.

### FIELD OF THE INVENTION

The present invention relates to a novel drug delivery system comprising a drug(s)-protein-polymer triple conjugate. The triple conjugate employs a (i) protein moiety capable of binding selectively to a particular target site possessed by a cell/affected organ, (ii) a polymer moiety, covalently linked to the protein and (iii) an active drug moiety that includes one or more drug(s) covalently linked to either said polymer moiety or to a protein moiety. The conjugates of the present invention have target specificity and better selectivity to a defined population of cells/organs(s). The present invention further relates to methods of preparation and methods of treatment comprising administering said conjugate as a single unit. The conjugates of the present invention are usefully employed in therapeutic as well as non-therapeutic, e.g., diagnostic applications.

### BACKGROUND OF THE INVENTION

A number of methods for enhancing the activity and specificity of the drug compositions such as anti-proliferative compositions have been known in the art. In general, the desired result is to increase both the efficiency and specificity of the therapeutic agent.

One method of achieving such result has been receptor targeting.

However, one drawback of receptor targeting lies in the finite number of receptors on target cells. It has been estimated that the maximum number of receptors on a cell is approximately one million (Darnell, Lodish and Baltimore, Molecular Cell Biology (1986)). Thus, there is a maximum binding of one million drug ligand complexes to any given cell. Furthermore, the maximum number of specific receptors is much lower, for example, for a specific steroid, there are between ten thousand and one hundred thousand. Thus, attempts at receptor targeting wherein the drug is complexed with a ligand specific for a single receptor type will result in a maximum binding of less than about one hundred thousand complexes per cell.

The protein polymer conjugates have been well known in the art having anti-viral and/or anti-proliferative activity. Pegylated interferons are also commercially available such as Pegasys and Pegintron for anti Hepatitis C therapy. The co-administration of a drug moiety and a protein-polymer conjugate such as interferon conjugate (pegylated interferon) is also known in the art. For example, US Patent Publication US20070202078 discloses co-administration of Ribavirin (an anti-viral drug) and interferon conjugate. U.S. Pat. No. 6,908,611 discloses co-administration of Vitamin B12 compounds with interferon compounds to enhance the efficacy of interferon compounds in viral, proliferative or inflammatory diseases. U.S. Pat. No. 6,752,986 discloses co-administration of interferon-beta and Cobalamin drug conjugates for treating conditions characterized by cellular proliferation. US Patent Publication 20090068280 discloses a controlled release formulation comprising a microparticle comprising a biodegradable polymer and one or more interferon compounds which could be administered in combination with one or more drug(s).

Although drugs mentioned herein above and those available commercially show efficacy to some extent, their lack of selectivity for tumour cells over normal cells can lead to severe side effects. Furthermore, emergence of drug resistance remains a significant problem in the treatment of breast and prostate cancer. In the case of breast cancer, arrival of the selective oestrogen receptor (ER) antagonist tamoxifen contributed to a 28% reduction in mortality at 5 years (Jordan 2003). Even so, the prognosis for patients particularly with metastatic breast cancer is still poor, the survival rate at 5 years being under 20%. The central problem in cancer chemotherapy is severe toxic side effects of anticancer drugs on healthy tissues. Invariably the side effects impose dose reduction, treatment delay, or discontinuance of therapy.

Therefore, in view of the foregoing, there remains a pressing need to formulate an effective cancer drug of therapy regime that has lower adverse side effects of cancer chemotherapy on healthy organs, prolonged therapeutic activity and better efficacy.

### OBJECTS OF THE INVENTION

Accordingly, it is of great interest to the pharmaceutical industry and related fields to develop a drug delivery system which would be highly target specific and have better selectivity to a defined population of cells/organ(s).

Accordingly, in one aspect, the invention discloses a novel formulation based on New Drug Delivery System wherein there occurs a minimized uptake of an active drug, by normal cells and enhances the influx and retention of the drug in cancer cells or tissues. The formulation comprises a drug(s), protein, polymer conjugate comprising of a protein capable of binding selectively to a particular target site possessed by a cell/tissue/organ(s), a polymer covalently linked to the protein and one or more drug molecules covalently linked either to said protein or to polymer in the form of a single pharmaceutical formulation.

It is an object of the invention to provide a pharmaceutical formulation which is administered as a single unit dose having better target specificity and selectivity.

It is an object of the invention to provide an advantage of lesser dose and reduced frequency of the administration of a drug in comparison to individual use of the drug or known drug-polymer or known protein polymer conjugates.

It is an object of the invention to provide the advantages of improved bio-availability of the drug molecule and reduction in number of side effects due to targeted delivery and sustained release.

It is an object of the invention to provide a pharmaceutical formulation based on novel drug delivery system wherein the formulation comprises a drug(s)-protein-polymer triple conjugate comprising of: (i) a protein moiety capable of binding selectively to a particular target site possessed by a cell, (ii) a polymeric carrier covalently linked to said protein moiety and (iii) one or more drug molecules covalently linked to said polymeric carrier or to protein moiety, wherein said conjugate is administered to a subject in need as a single species of triple conjugate.

Another object of the invention is to provide a drug(s)-protein-polymer triple conjugate that delivers one or more drug(s) with better target specificity and selectivity to a defined population of cells/tissue/organ(s).

It is a further object of the invention to provide a pharmaceutical formulation comprising said triple conjugates along with pharmaceutically acceptable excipients and diluents.

It is an object of the invention to provide methods for the preparation of said triple conjugates.

It is yet another object of the invention, to provide said drug(s)-protein-polymer triple conjugate that are usefully employed in therapeutic as well as non-therapeutic, for example diagnostic, applications.

It is a further object of the invention to provide drug(s)-protein-polymer triple conjugate that requires a reduced dose of the said drug and hence a reduced frequency of the administration of the drug in comparison to individual use of the drug or known protein-polymer or drug-polymer conjugates.

### SUMMARY OF THE INVENTION

The present invention provides a pharmaceutical formulation based on novel drug delivery system comprising a drug(s)-protein-polymer triple conjugate comprising of:

(i) a protein moiety capable of binding selectively to a particular target site possessed by a cell/organ(s),
(ii) a polymer moiety, covalently linked to the protein,
(iii) an active drug moiety, wherein the drug moiety comprises of at least one of active drug (s) covalently linked to either the protein moiety or to the polymer moiety.

In an embodiment of the invention, the protein moiety, the polymer moiety and the active drug moiety are linked to each other in any order.

In another embodiment of the invention, the conjugate is administered to a subject in need thereof as a single species/unit.

In an embodiment of the invention, the conjugate can also be formed by interchangeable binding between all the three moieties drug, polymer and protein depending upon available reactive group in each of these. The conjugate of the present invention have target specificity and better selectivity to a defined population of cells/organs and helps in reducing dose of active moiety additionally is helpful in drastic reduction in ADRs.

The present invention further relates to the processes for the preparation of said triple conjugate. The conjugate of the present invention are usefully employed in therapeutic as well as non-therapeutic, e.g., diagnostic applications.

In an embodiment of the present invention, the polymer moiety is present in an amount from about 60% to about 85%. The protein moiety may be present in an amount about 6% to about 12%. The active drug may be present in an amount 10% to about 20%.

In one embodiment, the molecular weight of the triple conjugate is from about 10 KDa to about 50 Kda.

In another embodiment, the molecular weight of the polymer moiety is from 0.2 KDa to about 45 Kda.

The polymer moiety is selected from a group comprising of polyalkylene glycols, polyethylene glycols (PEG), monomethoxypoly (ethylene glycols), monohydroxypoly (ethylene glycols), polyalkylene oxides, polyoxiranes, polyolefinic alcohols, polycarboxylates, polyvinylpyrrolidones, poly(oxyethyleneoxymethylenes), poly(amino acids), polyacryloylmorpholines, copolymers of amides, alkylene oxides, dextrans, hyaluronic acids, polyacrylamides, carbohydrate-based polymers, polynucleotides or any combination thereof.

In a further embodiment, the pharmaceutical formulation further comprises a carrier. The carrier is present in an amount from about 0.01 ml to about 0.5 ml.

In a further embodiment, the pharmaceutical formulation further comprises an activation agent. The activation agent is present in an amount from about 0.01 ml to about 0.5 ml.

The active drug is selected from the group including a cytotoxic drug, anti-viral drug, anti-neoplastic drug, anti-inflammatory drug, antibiotic, analgesic drug, drug acting on CNS, CVS, proton pump inhibitor or any combination thereof.

In one embodiment, the active drug moiety is an anticancer drug selected from a group comprising of anti-metabolites masquerade as purines, cisplatin, carboplatin, oxaliplatin, mechlorethamine, cyclophosphamide, chlorambucil, ifosfamide, Vincristine, Vinblastine, Vinorelbine, Vindesine, podophyllotoxins, etoposide teniposide, docetaxel, paclitaxel, irinotecan, topotecan, actinomycin, anthracyclines, doxorubicin, daunorubicin, valrubicin, idarubicin, epirubicin, bleomycin, plicamycin, mitomycin, dactinomycin, cytarabine, bortezomebe, fludarabine, clatribine, Gemcitabi, Methotrexate, 5-fluro uracil, Amscrine, Cladribine, Carmustine or pharmaceutically acceptable salts thereof.

The protein moiety is selected from the group comprising of immunoglobulins, glycoproteins, antibodies, polypeptides, enzymes, peptides, Interferon (INF), interleukins, hormones, somatomedins, erythropoietin, pigmentary hormones, hypothalamic releasing factors, antidiuretic hormones, prolactin, chorionic gonadotropin, follicle-stimulating hormone, thyroid-stimulating hormone or tissue plasminogen activator.

In one embodiment, the protein moiety is an Interferon (INF). The Interferon (INF) can be selected from INFα-2a, INFα-2b or INF-γ.

In a preferred embodiment, the INF is present in an amount about 0.001 ml to about 1.0 ml. The Interferon can be of 1 to 25 MIU potency.

In a preferred embodiment, the polymer moiety is PEG. In a further embodiment, the polymer moiety is present in an amount from about 0.5 ml to about 1.0 ml.

In a preferred embodiment, the active drug is docetaxel or a pharmaceutically acceptable salt thereof. In a further embodiment, the active drug is present in an amount from about 10 mg to about 40 mg.

In another embodiment, a method of preparation of a pharmaceutical formulation comprising triple conjugate including a polymer moiety, a protein moiety, and one or more active drug moiety is provided. The method comprises the steps of:

(ii) preparing a complex of the polymer moiety and the protein moiety by addition of the polymer moiety to the protein moiety to obtain a polymer-protein complex following addition of said drug moiety; or
(ii) adding the polymer moiety, the protein moiety and the drug moiety simultaneously to facilitate a conjugation.

In an embodiment of the invention, the conjugation is facilitated under an inert gas atmosphere and under constant stirring condition.

In an embodiment of the invention, the polymer moiety is activated by employing one or more activation agents wherein the activation enables the polymer to bind with the protein moiety or drug moiety or both.

In one embodiment, the activation of the polymer occurs when the polymer is added to the protein along with the activation agent. The activation of the polymer occurs when the polymer is added to the activation agent for a period ranging from 0.8 hrs to 24 hrs. The activation of the polymer occurs when the polymer is added to the activation agent for a period ranging about 2 hrs to about 16 hrs.

In a preferred embodiment, the conjugation occur for about 0.5 hrs to about 48 hrs.

In an embodiment, the activation of the polymer occurs when the polymer is added to the activation agent for a period ranging from 0.8 hrs to 24 hrs.

In an embodiment, the drug is optionally dissolved in one or more carriers before the drug is added to the polymer or to the polymer-protein complex. The carrier is selected from the group comprising of ethylene glycols, diethyleneglycol mono ethyl ether, polyalkylene oxides, polyoxiranes, polyolefinic alcohols, polycarboxylates, poly vinylpyrrolidones, poly xyethyleneoxymethylenes, polyamino acids, polyacryloylmorpholines, copolymers of amides, alkylene oxides, dextrans, hyaluronic acids or polyacrylamides preferably diethyleneglycol monoethyl ether.

In the most preferred embodiment, the drug is docetaxel or a pharmaceutically acceptable salt thereof.

The present invention further provides a method of treating a disease condition selected form the group comprising eoplastic diseases, autoimmune diseases, GERD, Ulcer, Autoimmune conditions, Diabetes, Genetic conditions, Viral/Bacterial/Parasitic Infections, Worm conditions, Physical conditions, Prion diseases, Nutritional deficiencies, Vitamin/Mineral deficiencies, Mitochondrial diseases, Accidents, Sexually Transmitted Diseases, Pregnancy Conditions, Breastfeeding Conditions, Birth defects, Male/Female/Infant/childhood/Adolescent conditions, Immune disorders, Balance disorders, Pain, Systemic disorders, Blood conditions, Blood vessel conditions, Nerve conditions, Muscle conditions, Heart conditions, Back/Neck/Spinalcord conditions, Eye conditions, Brain conditions, Mental conditions, Nose conditions, Mouth conditions, Dental conditions, Foot/Leg/Knee conditions, upper limb condition, Shoulder conditions, Ear conditions, Lung conditions, Liver conditions, Kidney conditions, Gall bladder conditions, Pancreas conditions, Digestive conditions, Prostate conditions, Male genital conditions, Obstetrical conditions, Gynaecological conditions, Thyroid disorders, Hearing disorders, by administering a therapeutically effective amount of the pharmaceutical formulation to a subject in need thereof.

In a preferred embodiment, the pharmaceutical formulation of the present invention is administered through a parenteral route.

A further embodiment provides a use of the pharmaceutical formulation of the present invention for preparation of a medicament for treating a disease condition selected from the group comprising neoplastic diseases, autoimmune diseases, GERD, Ulcer, Autoimmune conditions, Diabetes, Genetic conditions, Viral/Bacterial/Parasitic Infections, Worm conditions, Physical conditions, Prion diseases, Nutritional deficiencies, Vitamin/Mineral deficiencies, Mitochondrial diseases, Accidents, Sexually Transmitted Diseases, Pregnancy Conditions, Breastfeeding Conditions, Birth defects, Male/Female/Infant/childhood/Adolescent conditions, Immune disorders, Balance disorders, Pain, Systemic disorders, Blood conditions, Blood vessel conditions, Nerve conditions, Muscle conditions, Heart conditions, Back/Neck/Spinalcord conditions, Eye conditions, Brain conditions, Mental conditions, Nose conditions, Mouth conditions, Dental conditions, Foot/Leg/Knee conditions, upper limb condition, Shoulder conditions, Ear conditions, Lung conditions, Liver conditions, Kidney conditions, Gall bladder conditions, Pancreas conditions, Digestive conditions, Prostate conditions, Male genital conditions, Obstetrical conditions, Gynaecological conditions, Thyroid disorders, Hearing disorders or a combination of disease thereof, by administering a therapeutically effective amount of the pharmaceutical composition to a subject in need thereof.

### BRIEF DESCRIPTION OF THE DRAWINGS

The embodiments herein will be better understood in terms of their characteristics and effectiveness from the following detailed description with reference to the figures depicting various test results:

FIG. 1 illustrates a Comparative IR characterization of an embodiment of the present invention (VRP007)

FIG. 2 illustrates a Comparative NMR characterization of an embodiment of the present invention (VRP007).

FIG. 3 illustrates a Thin Layer Chromatography characterization: The Rf value of a Formulation 6 (one of the embodiments of the invention where Drug is Docetaxel, Protein is Interferon alpha 2a and Polymer is PEG 400) was 0.63 in comparison to 0.37 (Drug), 0.48 (Protein) and 0.42 (Polymer)

FIG. 4: illustrates stability study of the Formulation 6

FIG. 5A through FIG. 5B illustrate a Percentage inhibition of cell count in HER2 over expressed cell line (MCF 7) by different concentrations of all comparison groups at different time intervals.

FIG. 6 illustrates number of viable cells count including proliferative cells after incubation of different drug concentrations at different time intervals.

FIG. 7 illustrates a cell viability study.

FIG. 8 illustrates an agarose gel image indicating comparative DNA damage of an embodiment of the present invention and others.

FIG. 9 illustrates an In Vitro Release study of an embodiment of the present invention.

FIG. 10 illustrates a comparative percentage of tumor growth in DMBA induced breast cancer model.

FIG. 11 illustrates a comparative Effect of Different Comparative groups on tumor volume (cm) Reduction in Breast cancer Model

FIG. 12 illustrates a comparative reduction in adverse effects.

FIG. 13 illustrates a comparative Reduction in TNF alpha levels in tumor induced breast cancer model.

### DETAILED DESCRIPTION OF THE INVENTION

The embodiments herein and the various features and advantageous details thereof are explained more fully with reference to the non-limiting embodiments that are illustrated in the accompanying figures & tables and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

In one aspect, the present invention relates to a pharmaceutical formulation based on novel drug delivery system comprising a drug(s)-protein-polymer triple conjugate comprising of:

(i) a protein moiety capable of binding selectively to a particular target site possessed by a cell/organ(s),
(ii) a polymer moiety, covalently linked to the protein,
(iii) an active drug moiety, wherein the drug moiety comprises of at least one of active drug (s) covalently linked to either the protein moiety or to the polymer moiety,
wherein the protein moiety, the polymer moiety and the active drug moiety are linked to each other in any order, and
wherein the conjugate is administered to a subject in need thereof as a single species In another embodiment, the formulation optionally comprises of one or more carriers to facilitate binding of the active drug to the polymer moiety. In another embodiment the polymer moiety is either activated or activated using activation agent during the course of conjugation.
For purposes of different embodiments and aspects of the present invention, the term “protein” shall be understood to encompass not only proteins, but also immunoglobulins, glycoproteins, antibodies, polypeptides, enzymes, peptides and the like which are target specific. Proteins, polypeptides and peptides of interest include, but are not limited to, hemoglobin, serum proteins such as blood factors including Factors VII, VIII, and IX; immunoglobulins, cytokines such as interleukins, i.e. IL-1 through IL-13, interferon-alphas, interferon-betas, interferon-gamma, lectins, sugar binding proteins, glycoproteins, SUMO proteins preferably interferons, lectins as described in more detail below, colony stimulating factors including granulocyte colony stimulating factors, platelet derived growth factors and phospholipase-activating protein (PLAP). Other proteins of general biological or therapeutic interest include insulin, plant proteins such as lectins and ricins, tumor necrosis factors and related proteins, growth factors such as transforming growth factors, such as TGFa's or TGFβ's and epidermal growth factors, hormones, somatomedins, erythropoietin, pigmentary hormones, hypothalamic releasing factors, antidiuretic hormones, prolactin, chorionic gonadotropin, follicle-stimulating hormone, thyroid-stimulating hormone, tissue plasminogen activator, and the like.

In one aspect, the Immunoglobulins of interest include IgG, IgE, IgM, IgA, IgD and fragments thereof.

In another aspect, additional suitable therapeutic proteins include monoclonal and polyclonal antibodies, single-chain antibodies, other antibody fragments, analogs and derivatives. Therapeutic poly nucleotides, including antisense oligonucleotides, aptamers and therapeutic genes also can be delivered using the methods and compositions of the invention.

In certain aspect, some proteins such as the interleukins, interferons and colony stimulating factors also exist in non-glycosylated form, usually as a result of using recombinant techniques. The non-glycosylated versions are also among the proteins of the present invention.

In another aspect, the enzymes of interest include carbohydrate-specific enzymes, proteolytic enzymes, oxidoreductases, transferases, hydrolases, lyases, isomerases and ligases.

The proteins or portions thereof can be prepared or isolated by using techniques known to those of ordinary skill in the art such as tissue culture, extraction from animal sources, or by recombinant DNA methodologies. Transgenic sources of the proteins, polypeptides, amino acid sequences and the like are also contemplated. Such materials are obtained from transgenic animals, i.e., mice, pigs, cows, dogs etc., wherein the proteins are expressed in milk, blood or tissues. Transgenic insects and baculovirus expression systems are also contemplated as sources. Moreover, mutant versions of proteins, such as mutant interferons are also within the scope of the invention.

Other proteins of interest are allergen proteins such as ragweed, Antigen E, honeybee venom, mite allergen, and the like.

In a preferred embodiment, the protein is interferon as described herein below. The foregoing is illustrative of the proteins which are suitable for the present invention. When the protein is an interferon (IFN), it will be understood that present invention includes interferons (IFN's) of all types as well as all subtypes of the foregoing. The term “interferon” as used herein means the family of highly homologous species-specific proteins that inhibit viral replication and cellular proliferation and modulate immune response. Human interferons are grouped into three classes based on their cellular origin and antigenicity: α-interferon (leukocytes), β-interferon (fibroblasts) and γ-interferon (B cells). Recombinant forms of each group have been developed and are commercially available. Subtypes in each group are based on antigenic/structural characteristics.

In one embodiment, the formulation employees INF as the protein moiety to target a intended receptor.

The interferons can also be prepared using recombinant techniques such as those using synthetic genes expressed in E. coli and other techniques known to those of ordinary skill in the art. Alpha and gamma interferons are preferred for the conjugates of the present invention according to embodiments herein.

At least 24 interferon alphas (grouped into subtypes A through H) having distinct amino acid sequences have been identified by isolating and sequencing DNA encoding these peptides (See also Viscomi, 1996 Biotherapy 10:59-86).

In certain aspect of the invention, both naturally occurring and recombinant interferons may be employed in the practice of the invention. It is also understood that the recombinant techniques could also include a glycosylation site for addition of a carbohydrate moiety on the recombinantly-derived polypeptide.

In another embodiment, the term “polymeric carrier” or “polymer” or “carrier” include one or more polyalkylene glycols (including, but not limited to, one or more poly(ethylene glycols) (PEG)), one or more monomethoxypoly(ethylene glycols), diethyleneglycol mono ethyl ether and one or more monohydroxypoly(ethylene glycols)), one or more polyalkylene oxides, one or more polyoxiranes, one or more polyolefinic alcohols, e.g., polyvinyl alcohol, one or more polycarboxylates, one or more poly(vinylpyrrolidones), one or more poly(oxyethyleneoxymethylenes), one or more poly(amino acids), one or more polyacryloylmorpholines, one or more copolymers of one or more amides and one or more alkylene oxides, one or more dextrans, one or more hyaluronic acids, one or more polyacrylamides, one or more carbohydrate-based polymers, polynucleotides and the like. Those of ordinary skill in the art will recognize that the foregoing list is merely illustrative and that all polymer materials having the qualities described herein are contemplated.

The polymer need not have any particular molecular weight, but it is preferred that the range of the molecular weight is ≧0.2 to ≦50 Kda, preferably ≧0.2 to ≦20 KDa according to an embodiment herein.

In another aspect of the invention, the protein moiety, the polymer moiety and the drug moiety bind to each other in any order.

In an yet another embodiment, the present invention provides a drug(s)-protein-polymer triple conjugate for targeted delivery of one of more active drug(s), employed in the triple conjugate, that has better specificity and selectivity to the defined population of cells including but not limited to, for example, tumor cells.

In an embodiment of the invention, the active drug molecule can be but not limited to, for example, cytotoxic drugs, anti-viral drugs, anti-neoplastic drugs, anti-inflammatory drugs, antibiotics, analgesic, drugs acting on CNS, CVS, proton pump inhibitors and all other drug categories defined in texts.

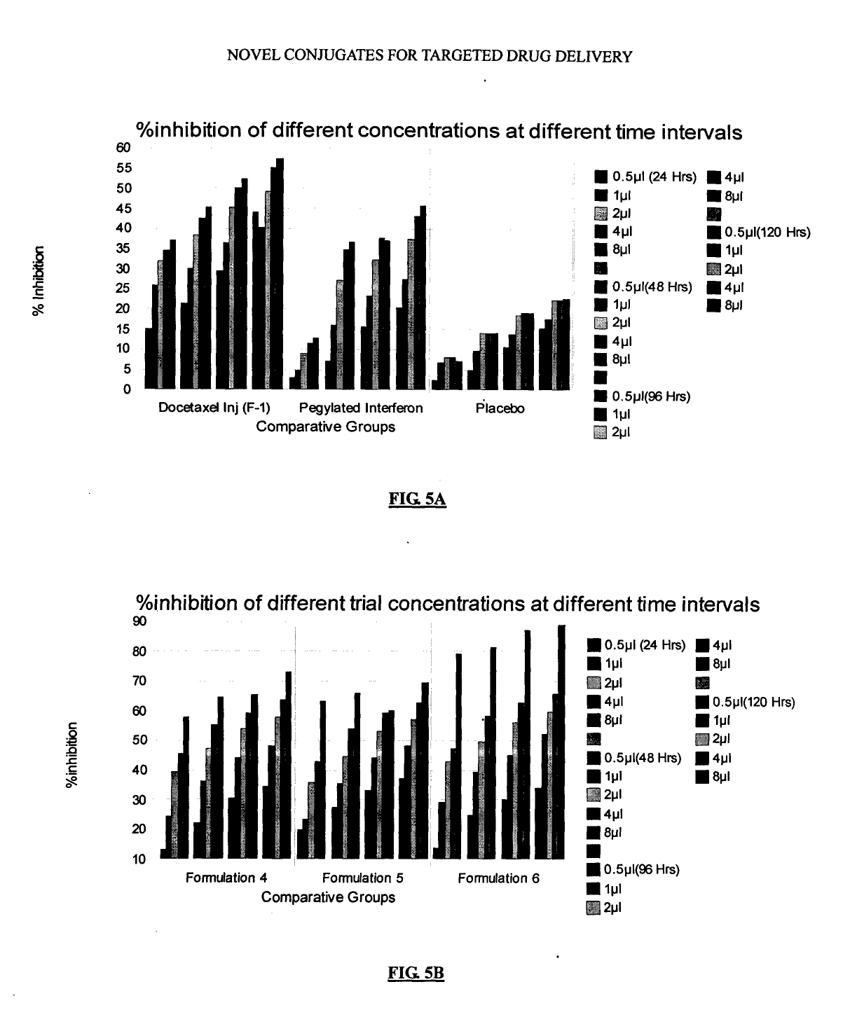

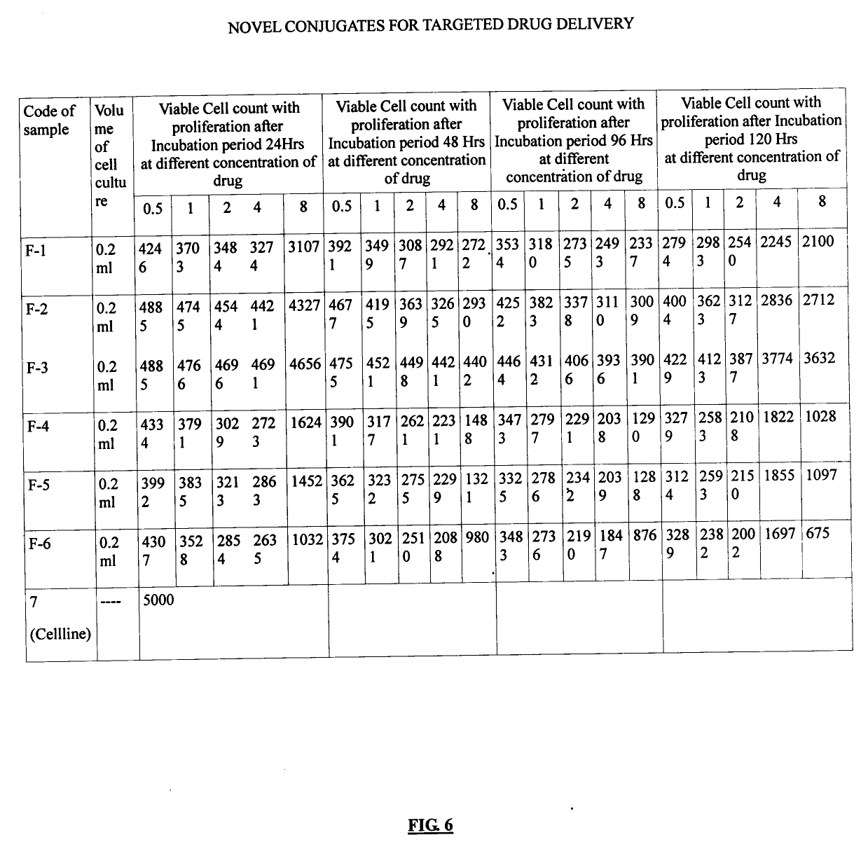

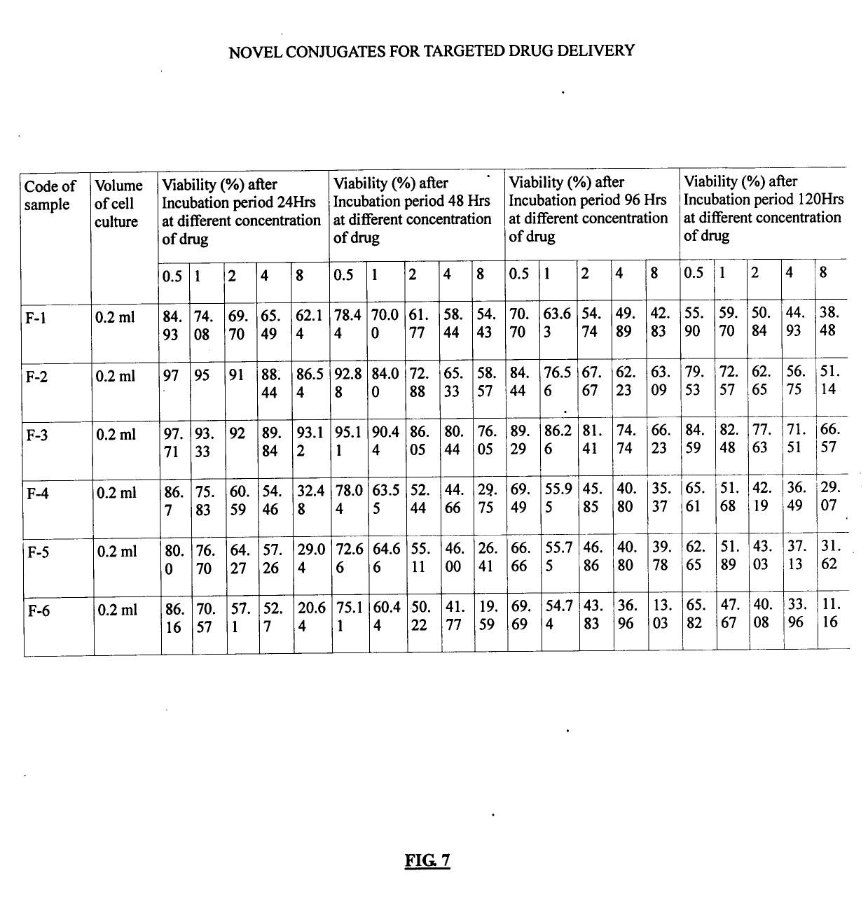

According to a preferred embodiment, the protein moiety is preferably a class of interferons given the fact that HER2 are over expressed in breast cancer, ovarian cancer which are targeted with INF-α2a , in prostate cancer INF-γ interacts with HER-2 and in gastric and head and neck carcinomas, INF-α2b has selective binding affinity. FIG. 5A through 5B illustrates a Percentage inhibition of cell count in HER2 over expressed cell line (MCF 7) by different concentrations of all comparison groups at different time intervals.

In a preferred embodiment, the active drug moiety is cytotoxic drug having specificity to particular tumor/tissue/cell line. The tumor/tissue/cell line comprises but not limited to Anti-metabolites masquerade as purines, alkyl agents such as cisplatin, carboplatin, oxaliplatin, mechlorethamine, cyclophosphamide, chlorambucil, ifosfamide, plant alkaloids including vinca alkaloids such as Vincristine, Vinblastine, Vinorelbine, Vindesine, podophyllotoxins: etoposide and teniposide, taxanes such as docetaxel, paclitaxel, topoisomerase inhibitors such as irinotecan and topotecan, cytotoxic antibiotics such as actinomycin, anthracyclines, doxorubicin, dactinomycin, cytrabine, bortezomib, gemcitabine, daunorubicin, valrubicin, idarubicin, epirubicin, bleomycin, fludarabine, clatribine, Gemcitabi, Methotrexate, 5-fluro uracil, Amscrine, Cladribine, Carmustine plicamycin, mitomycin and the like.

In another embodiment of the invention, the formulation comprising the drug(s)-protein-polymer triple conjugate is useful in treating or preventing neoplastic diseases, autoimmune diseases, GERD, Ulcer, Autoimmune conditions, Diabetes, Genetic conditions, Viral/Bacterial/Parasitic Infections, Worm conditions, Physical conditions, Prion diseases, Nutritional deficiencies, Vitamin/Mineral deficiencies, Mitochondrial diseases, Accidents, Sexually Transmitted Diseases, Pregnancy Conditions, Breastfeeding Conditions, Birth defects, Male/Female/Infant/childhood/Adolescent conditions, Immune disorders, Balance disorders, Pain, Systemic disorders, Blood conditions, Blood vessel conditions, Nerve conditions, Muscle conditions, Heart conditions, Back/Neck/Spinalcord conditions, Eye conditions, Brain conditions, Mental conditions, Nose conditions, Mouth conditions, Dental conditions, Foot/Leg/Knee conditions, upper limb condition, Shoulder conditions, Ear conditions, Lung conditions, Liver conditions, Kidney conditions, Gall bladder conditions, Pancreas conditions, Digestive conditions, Prostate conditions, Male genital conditions, Obstetrical conditions, Gynaecological conditions, Thyroid disorders, Hearing disorders that express an addressable receptor and respective drug to be delivered in order to control specific disease condition.

In a further embodiment of the present invention wherein the active drug employed in the triple conjugate is a cytotoxic drug, the drug(s)-protein-polymer triple conjugate is useful for treating or preventing neoplastic diseases or autoimmunities, or allergies or any condition that requires elimination of specific cell populations that express an addressable receptor, comprising single administration of a nontoxic, therapeutically effective amount of the triple conjugate as a single species to a subject, in need thereof.

In another embodiment, the drug, to be carried to an intended target site, is a cytotoxic drug. The drug(s)-protein-polymer triple conjugate has a lesser toxicity of the cytotoxic drug in comparison to the toxicity of the cytotoxic drug administered individually.

In another embodiment of the invention wherein the active drug used in the triple conjugate is a cytotoxic drug, the formulation of the drug(s)-protein-polymer triple conjugate enhances the immune power of body against tumor cells besides targeting the drug. In a preferred embodiment, the drug of cytotoxic nature is selected from the group comprising of taxens including docetaxel or a pharmaceutically acceptable salt thereof.

According to one embodiment of the present invention, the invention comprises a formulation based on novel drug delivery system for targetted delivery of at least on of active drug that includes a drug(s)-protein-polymer triple conjugate. The triple conjugate comprising:

(a) A protein moiety that includes one or more proteins wherein the protein moiety is capable of binding selectively to a particular target site possessed by a cell/tissue(s)/organ(s), and
(b) A polymer moiety covalently linked to the protein moiety, and
(c) An active drug moiety comprising of at least of the active drug covalently linked to either to the protein moiety or to the polymer moiety.
The active drug moiety may also be covalently linked or to an associated carrier to the drug moiety and then bind to the protein moiety or the drug moiety. The drug moiety is carried in to a the target site(s).

In an embodiment of the invention, the active drug moiety is optionally dissolved in a polymeric/non polymeric carrier prior to binding to the polymer moiety to form a conjugate which is internalized by the cell/organ. The protein included in the protein moiety is target binding site specific and directs the triple conjugate to the target/tumor site. Conjugate after binding to the target site is either internalized or engulfed or split by cellular enzymes to releases one or more active drug(s), targeting defined population of cells/tissue/organ, including but not limited to, for example, tumor cells.

In an embodiment, the polymer is procured which is pre-activated. Alternatively the polymer is activated simultaneously during the conjugation process by addition of the polymer to the protein and the protein. The conjugation may be any processes known to a person skilled in art including but not limited to biotinylation using Sulpho NHS biotin.

In a further embodiment, wherein the active drug to be carried to an intended target site is a cytotoxic drug, the formulation of the drug(s)-protein-polymer triple conjugate is preferably of reduced cytotoxicity to normal cells as compared to the active constituent (such as active drug or the protein) in their free form. The high specificity and reduced cytotoxicity of the formulation is obtained through Enhanced Permeability and Retention Effect.

Intracellular release of the cytotoxic drug as depot in cytotoxic form is accomplished by cellular enzymes, preferably enzymes expressed in tumor cells.

In a further embodiment, wherein the active drug to be carried to an intended target site is a cytotoxic drug, the drug(s)-protein-polymer triple conjugate having cytotoxicity comprises a protein which is capable of selectively binding to a particular target site possessed by a cell to be killed, a polymer which has cytotoxic drug(s) linked to its side chains and a reactive group at its terminal, both being covalently bound to each other, and a process for the preparation thereof.

According to yet another embodiment, the molecular weight of the triple conjugate is in the range of <50 KDa. According to yet another embodiment the molecular weight is preferably less than 25 KDa.

In another embodiment, a method of preparation of a pharmaceutical formulation comprising triple conjugate including a polymer moiety, a protein moiety, and one or more active drug moiety is provided. The method comprises the steps of: (i) preparing a complex of the polymer and the protein by addition of the polymer to the protein to obtain a polymer-protein complex, (ii) optionally activating the polymer by employing one or more of an activation agent, and (iii) adding the drug to the polymer-protein complex to facilitate a conjugation under an inert gas atmosphere and under constant stirring condition. The drug moiety is optionally dissolved in carrier solvent prior to adding in protein polymer complex. The activation enables binding of the drug and the protein to the polymer. In a preferred embodiment, the activation agent is Sulphur NHS biotin.

In a certain aspect of preparation of the triple conjugate, the polymer moiety employed therein is selected from a group comprising polymers of PEG having molecular weight <50 KDa, preferably <50 KDa, still preferably <5 KDa, more preferably <2 KDa. As described herein before, the polymer is activated to facilitate opening of its binding sites and consequent binding with the protein moiety and the drug moiety. The activation of polymer moiety is achieved by using different activation mechanism known to a person skilled in art including biotinylation. The activation of the polymer moiety though biotinylation is done employing an activation agent such as Sulpho NHS biotin. The carrier which may be of polymeric nature or otherwise, is selected from a group comprising T-80, PG, Ethyl alcohol, or pharmaceutically acceptable solvents. The polymeric carrier is preferably diethylene glycol mono ethyl ether. The protein moiety preferably comprises of INF α-2a, INF α-2b, or INF gamma (INFγ) in an amount about 0.001 ml to 1.0 ml of 1 to 25 MIU potency.

In one preferred embodiment, the protein moiety is mixed with PEG preferably with PEG 400 which is pre-activated using activation agent, NHS biotin, under condition of stirring in an inter gas atmosphere wherein the prevailing temperature is about 1° C. to about 8° C. for a period of 0.08 to 24 hrs preferably, 2 hrs to 16 hrs. The solution thus obtained is kept for conjugation for about 0.08 hrs to about to 48 hr under condition of stirring for obtaining the triple conjugate.

In an illustrative example of experimental set up, a comparator group Formulation 1 includes docetaxel trihydrate dissolved in the polymeric carrier of tween-80. A formulation of comparator group Formulation 2 includes pre-activated PEG with INFα-2a and a Formulation 3 is placebo.

In an preferred embodiment, the pharmaceutical formulation that includes the triple conjugate comprises about 60% to about 85% of the polymer moiety, about 6% to about 12% of the protein moiety and about 10% to about 20% of the active drug moiety with or without excipient/carrier.

In a preferred embodiment, Formulation 4 comprising the triple conjugate includes about 0.8 ml of PEG 400 as the polymer moiety, about 0.1 ml of INF γ as the protein moiety, about 0.1 ml of diethyleneglycolmono ethyl ether (DEGMEE as carrier and about 20 mg of active drug as Docetaxel Trihydrate. The formulation 4 optionally contains activation agent in amount about 0.05 mg of sulpho NHS biotin. A Formulation 5 comprising the triple conjugate includes about 0.8 ml of PEG 400 as the polymer moiety, about 0.1 ml of INFα-2b as the protein moiety, about 0.1 ml of diethyleneglycolmono ethyl ether and about 20 mg of active drug as Docetaxel Trihydrate. Formulation 5 optionally contains activation agent Sulpho NHS biotin in amount about 0.05 mg. Formulation 6 comprising the triple conjugate includes about 0.9 ml of PEG 400 as the polymer moiety, about 0.075 ml of INF α-2a as the protein moiety, about 0.1 ml of diethyleneglycolmono ethyl ether as carrier and about 20 mg of active drug as Anhydrous Docetaxel. Formulation 7 (VRF007) comprising the triple conjugate includes PEG of molecular weight of about 43 KDa in an amount 0.5 ml, the carrier as DMI in an amount about 0.2 ml as carrier, the protein as INFα-2b in an amount of about 0.05 ml and the active drug as Docetaxel trihydrate equivalent to about 20 mg.

Formulation 4 is prepared by employing about 0.8 ml of PEG wherein the PEG 400 is activated using 0.05 mg of Sulpho NHS Biotin, about 0.1 ml of INFγ, a polymer in an amount about 0.1 ml, a active drug moiety in the form of Docetaxel Trihydrate equivalent to about 20 mg where the active drug moiety is first dissolved in the carrier base for about 30 min under an inert gas flushing and under cold room conditions to obtain a first solution. The activation of polymer moiety which is, PEG is carried out by employing about 0.05 mg of Sulpho NHS Biotin for about 2 hrs at cold room conditions and under an inert gas atmosphere to obtain a second solution. In a subsequent step, both the first solution and the second solution is mixed along with addition of INFγ maintaining a slow stirring condition for about 6 hrs to achieve final conjugation to obtain a triple conjugate under restricted processing condition.

Formulation 5 comprises of PEG 400 in an amount of 0.8 ml, Sulpho NHS Biotin in an amount of 0.05 mg, INFα-2b in amount of about 0.1 ml, carrier in an amount of about 0.1 ml, the drug moiety as Docetaxel Trihydrate equivalent to about 20 mg wherein 0.1 ml of active drug moiety is not pre-dissolved in the carrier wherein the carrier is diethyleneglycolmono ethyl ether and is directly added to about 0.8 ml of PEG by a simultaneous activation by employing about 0.05 mg Sulpho NHS biotin along with simultaneous addition of about 0.1 ml of INFα-2b for about 16 hrs at cold room conditions and under inert gas atmosphere to achieve final conjugation and to obtain the tripe conjugate under restricted processing condition.

Formulation 6 (F-6) comprises PEG 400 in an amount of about 0.9 ml, Sulpho NHS Biotin in an amount of about 0.05 mg, INFα-2A in amount of about 0.075 ml, active drug moiety as Anhydrous Docetaxel equivalent to about 20 mg wherein about 0.9 ml of PEG 400 activation is carried out by employing about 0.05 mg of Sulpho NHS Biotin for about 2 hrs at cold room conditions and under inert gas atmosphere and addition of about 0.1 ml of INFα2A along with direct addition of Docetaxel anhydrous of about 20 mg for about 16 hrs at cold room conditions and under inert gas atmosphere to achieve final conjugation to obtain the triple conjugate under restricted processing condition. The formulation 7 is prepared by following the same procedure as is used for preparing Formulation 5 and Formulation 6.

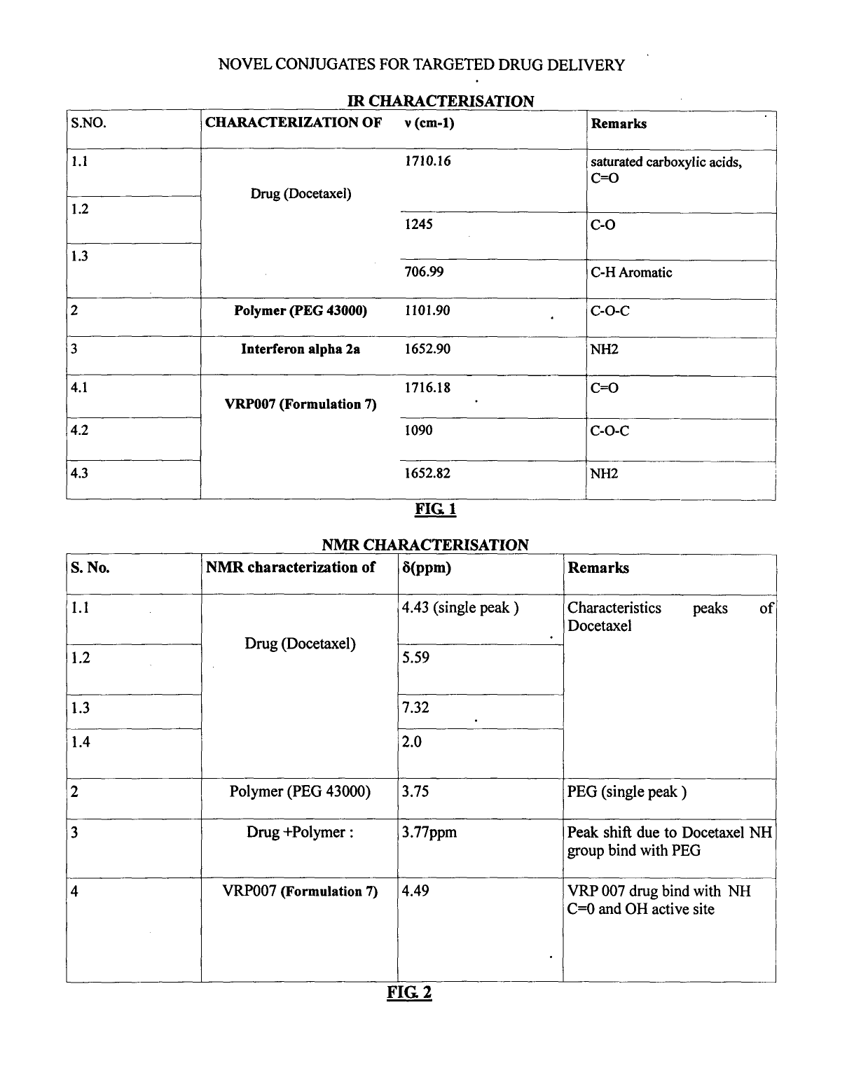

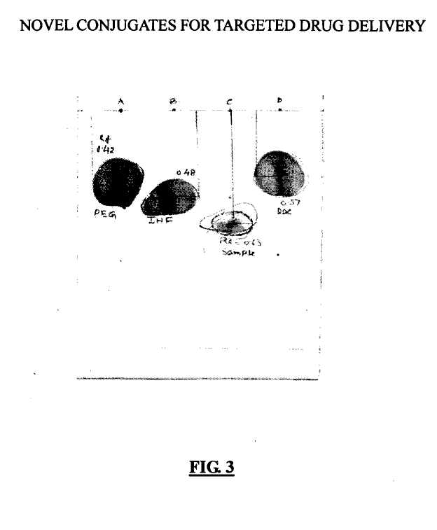

FIG. 1 and FIG. 2 illustrate IR and NMR characterization of a representative triple conjugate-VRP007 (VRP007 is equivalent to the aforementioned Formulation 7). The FTIR determination findings depict that for the development of VRP007 product, the functional groups of individual ingredient of VRP007 were determined. Individual Polymer shows the spectra at 1101.90 Cm⁻¹. At this spectra C—O—C functional group was found. There are several reports suggesting that at 1108 Cm⁻¹ C—O—C functional group is present where as single INF show functional group amide I at 1652.90 Cm⁻¹. Sharma et. al 2004, reported that amide I functional group show at 1650 Cm⁻¹. The single drug show band at 1710.16 Cm⁻¹, 1245 Cm⁻¹ and 706.99 Cm⁻¹. All most similar work reported by Yang et al DMI is co polymer which is chesterfield agent which show band at 2361 Cm⁻1. After preparation of formulation VRP 007 these peaks are shifted which is shown in the FIG. The NMR identification and findings in FIG. 2 concluded that a unique, trimer-structured, 43 KDa PEG was conjugated to interferon (IFN) by forming an amide bond to improve the pharmacokinetic properties and minimize the loss of IFN bio-activity. The drug has has C13 which contains hydroxyl group as well as amide and C═O groups. It has most affinity and chance to bind the PEG and carrier with —NH group. The drug shows a peak at 4.43 ppm and 3.3 ppm due to aromatic ring. When single peak of polymer is compared with polymer and drug, the peak of polymer was shifted around to 3.77 ppm. It means that —NH group of drug may be bound with polymer, so the shifting might have occurred. This can be deduced from a comparison of the NMR spectra of the VRP007 and drug that single peak at 4.43 ppm in drug is shifted to 4.49 ppm in VRP007 due to conjugation at —NH C=0 and —OH active sites of drug.

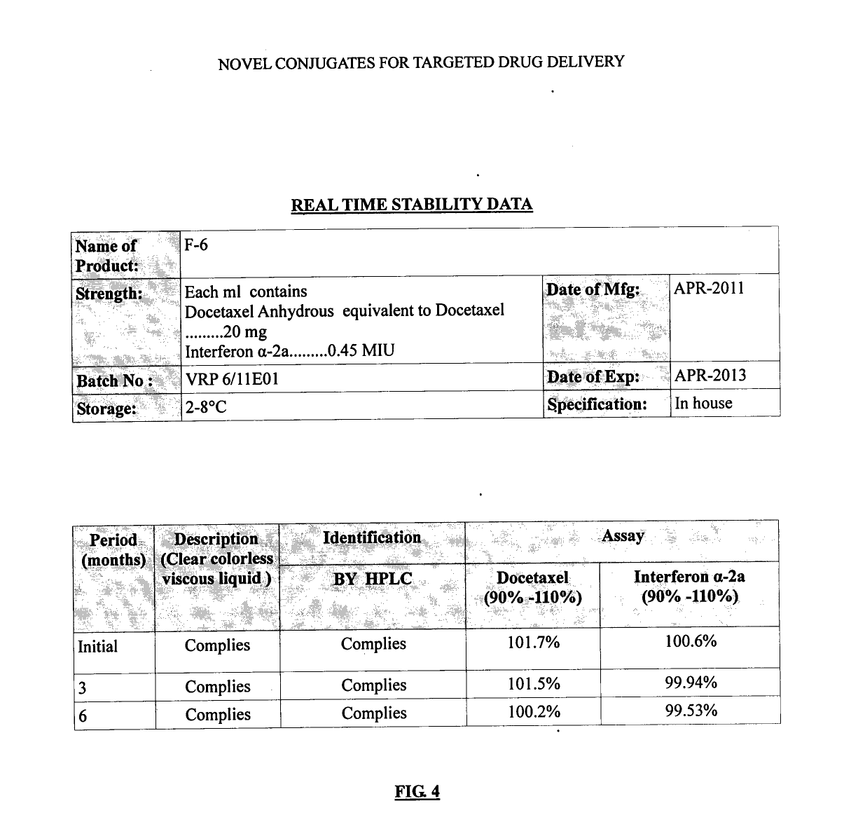

In FIG. 3 an illustration of a Thin Layer Chromatography Characterization of VRP007 is given: The Rf value of the triple conjugate, Formulation 6 (where Drug is Docetaxel, Protein is Interferon alpha 2a and Polymer is PEG 400) was 0.63 in comparison to 0.37 (Drug), 0.48 (Protein) and 0.42 (Polymer). Individual components (drug, protein, polymer) as well as Formulation 6 were run simultaneously. All the samples were run simultaneously on silca gel and relative mobility was measured. In this study relative mobility of Formulation 6 (F-6) was high due to higher polarity than all other components. The Rf was calculated according to formula: Distance travel of solvent/Distance travel of solute.

In another embodiment, the release of the active drug molecule is preferred because, as a rule, the low molecular weight drug molecules must interact with the target molecule in order to bring its pharmacological effectiveness into play. The drug(s)-protein-polymer triple conjugate of the invention represents a transport and/or depot form of the pharmaceutically and/or diagnostically active drug molecule, which thus reaches the target cells or the target tissue of the drug in targeted manner or in metered form.

In FIG. 4 an illustration is given to show a real time stability data for the Formulation 6 (F6) wherein all tests are carried out as per STP and it was found that F-6 is stable at 2° C. to 8° C. for 6 months, stability continued.

FIG. 5A through 5B illustrate a percentage inhibition of cell count in HER2 over expressed cell line (MCF 7) by different concentrations of all comparison groups at different time intervals. FIG. 6 illustrates number of viable cells count including proliferative cells after incubation of different drug concentrations at different time intervals. In this study, Sample 1 is docetaxel alone, Sample 2 is pegylated interferon, Sample 3 is placebo, Sample 4 is Formulation 4 (F4), Sample 5 is Formulation 5 (F5), Sample 6 is F-6. FIG. 7 illustrates a cell viability study wherein it is observed that number of viable cells is dependent upon initial drug concentration. Higher the concentration more is the killing. At 8 μl concentration, number of viable cells including proliferative cells has reduced to 20.64% as compared to control, indicating >80% killing. Killing is constant after incubation of 3-4 days and drastic decrease in cell count is not observed. In the study as represented in FIGS. 5A, 5B, FIG. 6 and FIG. 7, during the cell proliferation, cells were seeded in a 96-well plate at a density of 5000 cells/well and allowed to adhere for 24 hr prior to the assay. Cells were exposed to a series of formulations naming 1 to 6 at 37° C. After every 24 hr till 120 h of incubation, media was replaced with 100 μl of fresh media and 10 μl of MTT indicator dye ((15 mg/ml) was added to each well and the cells were incubated for another 2 h at 37° C. in the CO₂ incubator. Then 100 μl solubilization buffer was added to each well and agitated thoroughly to dissolve the formazan crystals. The plate was read on a micro plate reader at 600 nm. Cell viability, % inhibition and cell proliferation was calculated and presented in below table.

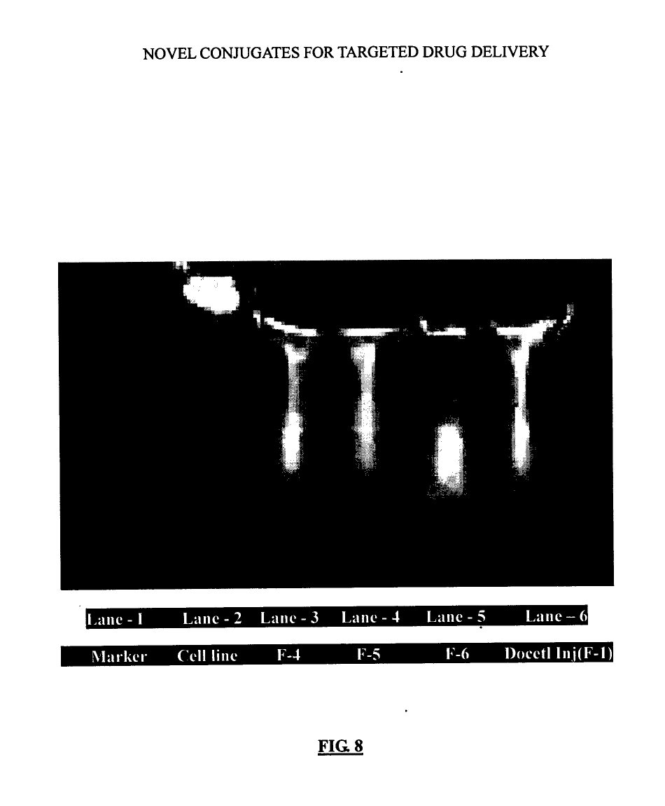

FIG. 8 illustrates an agarose gel electrophoresis image indicating comparative DNA damage between study formulations and an embodiment of the present invention. After treatment of cells with various docetaxel and various formulations of triple conjugate, cells were harvested. Cells were then suspended in a lysis solution containing 100 mM Tris-HCl (pH-8.5), 400 mM NaCl, 5 mM EDTA and 0.2% SDS and incubated overnight. After incubation, equal volume of chilled isopropanol was added to samples and centrifuged at 6000 rpm for 10 minute at 15° C. Then, the pellet was washed twice with 70% chilled ethanol and centrifuge at 6000 rpm for 5 minutes at 15° C. After washing, the DNA was air dried briefly (10 min), and then dissolved in 25 μl of TE buffer. DNA samples were analyzed by electrophoresis in a 0.8% agarose gel containing 10 mg/ml ethidium bromide and visualized under UV illumination. According to this study Lane 1: Marker; Lane 2: Control cell lines without treatment; Lane 3: Treated with F 4; Lane 4: Treated with F 5; Lane 5: Treated with F-6; Lane 6: Treated with Formulation 1 (Docetaxel Inj). The results showed maximum mobility occurring for F-6 that established high DNA damage.

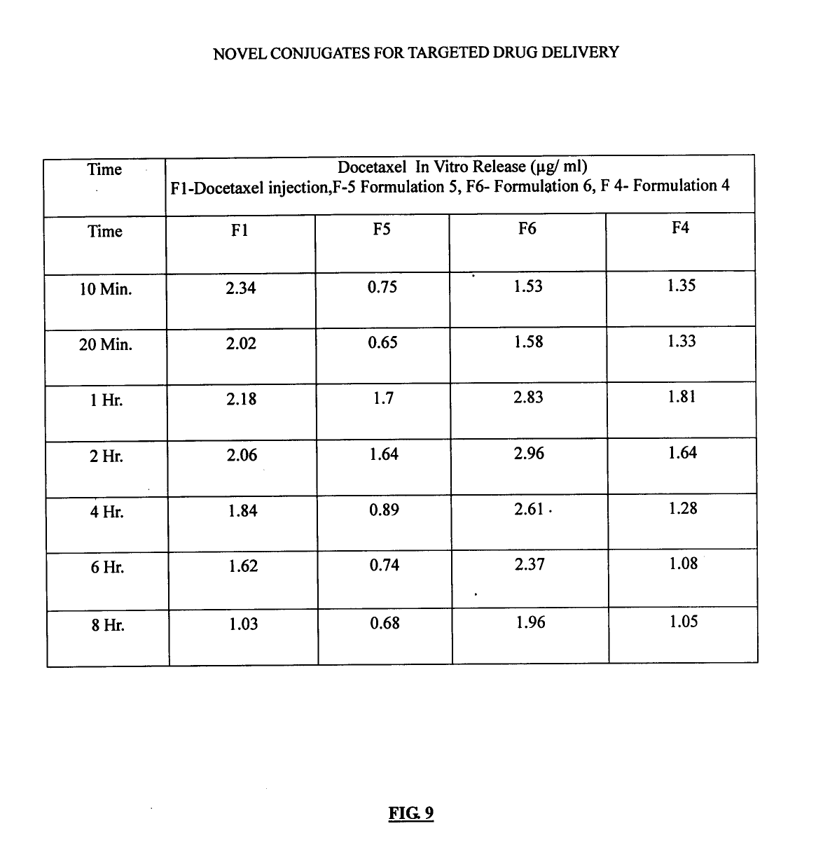

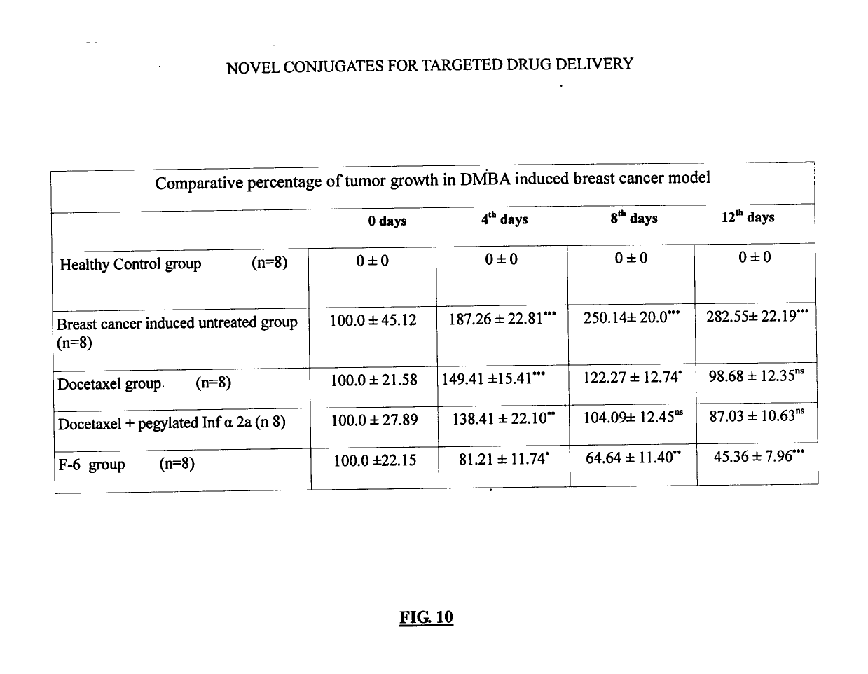

In a further embodiment of the invention, the drug(s)-protein-polymer triple conjugate has an improved bio-availability of the drug employed in the triple conjugate. FIG. 9 illustrates an in vitro release study of an embodiment of the present invention. During this study, 10 mg (0.5 ml) of each Formulation 1 (F1), Formulation 4 (F2), F5 and F6 were placed into a pre-swelled dialysis bag with a 12 KDa molecular weight cutoff and immersed into Dextrose, at 4° C. with gentle agitation. The incubation medium was sampled at various time points to monitor the active drug, that is, Docetaxel release rate. After sampling, equal volume of fresh dextrose was immediately added back to keep the constant volume of the incubation medium. The concentration of Docetaxel released from the each was expressed as μg/ml and plotted as a function of time. It was observed that F1 in the form of an injection achieved maximum drug concentration in 10 min and then the drug concentrations started falling. In F5, F6 and F4, depending upon the amount of conjugation achieved, maximum concentration was achieved in 1-2 hrs and then drug concentration reduced slowly indicating a sustained release effect of drug due to binding.

In an embodiment of the invention, the conjugating polymer may utilize any other groups, moieties, or other conjugated species, as appropriate to the end use application. By way of example, it may be useful in some applications to covalently bond to the polymer, a functional moiety imparting UV-degradation resistance, or antioxidation, or other properties or characteristics to the polymer. As a further example, it may be advantageous in some applications to functionalize the polymer to render it reactive or cross-linkable in character and to enhance various properties or characteristics of the overall conjugated material. Accordingly, the polymer may contain any functionality, repeating groups, linkages, or other constituent structures which do not preclude the efficacy of the conjugate for its intended purpose.

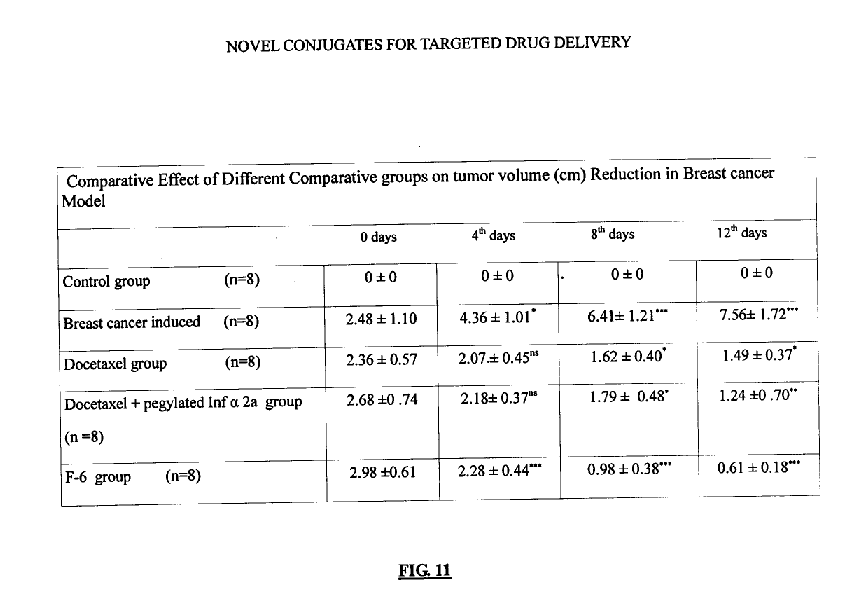

FIG. 10 illustrates a comparative percentage of tumor growth in DMBA induced breast cancer model in study titled “Comparative effect of Docetaxel Injection, docetaxel administered with pegylated interferon vs Triple conjugate, Formulation 6 in Breast cancer induced rat model”. FIG. 11 illustrates a comparative Effect of Different Comparative groups on tumor volume (cm) Reduction in Breast cancer Model. In the study as represented in FIG. 10 an FIG. 11, tumour induction was achieved in a female Sprague Dawley rats at the age of 8 weeks weighing 160-180 g and were gavaged with 60 mg dimethylbenz[a]anthracene (DMBA)/kg body weight, a dose sufficient to cause 100% tumour incidence in the control group over the course of the study as described by Whitsett T et al. The DMBA was dissolved in olive oil at a stock solution of 30 mg/ml. Doses administered were equivalent to standard human dose of 60 mg/m² of docetaxel. The data was compared between Docetaxel treated group vs. the F-6 treated group. All data are mean±SD. The Neuman Kaul test was performed for statistical significance between control vs breast cancer induced group as well Docetaxel vs. F-6.

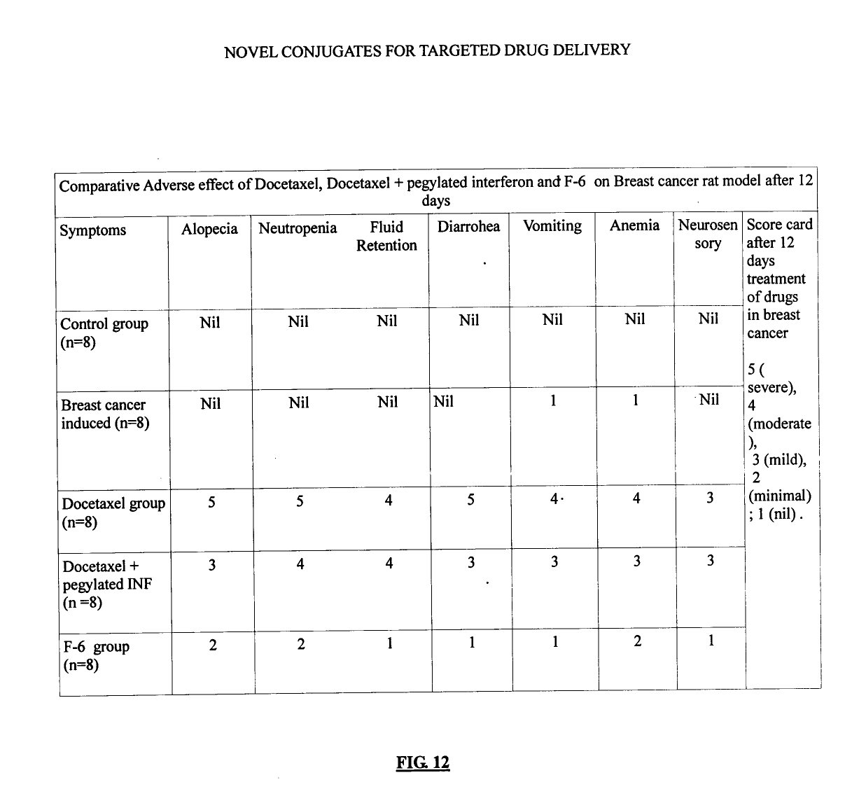

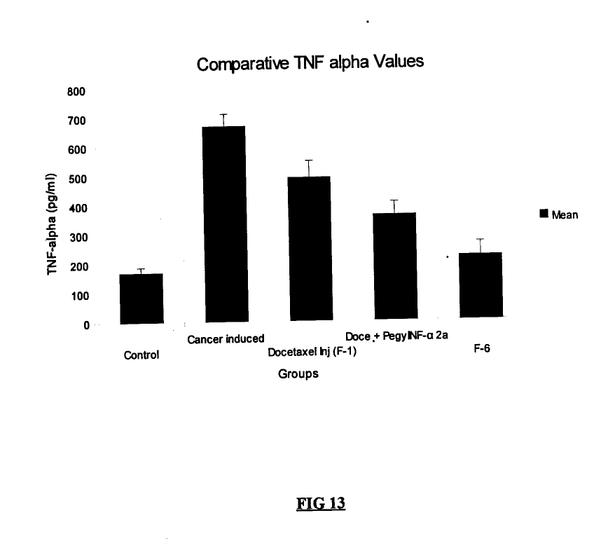

According to a certain aspect, the triple conjugate shows targeted delivery of the active drug in more precise location of the intended target site in a cell/tissue or organ thereby enabling a minimized uptake of the drug by normal cell and high uptake by diseased cells resulting in a lower adverse effect. FIG. 12 illustrates a Comparative reduction in adverse effects wherein there is a significantly increased (p<0.001) percentage of tumour in breast cancer induced group as compared to control. In case of Docetaxel, docetaxel plus pegylated interferon 2a (administered one after the other) and F6 drugs, the tumour was significantly inhibited by only F-6 treated group on 12^{th} day as compared to 0 day. There was significantly increased tumour volume in breast cancer induced group as compared control group. After treatment with respective drug, the tumour volume was significantly decreased in F-6 along with TNF alpha level in same group. All the drugs caused adverse effect in breast cancer model but in F-6 treated group, the adverse effect was significantly less in comparison to other drug treated groups due to targeted delivery of docetaxel. Further significant reduction in tumour volume by treatment with F-6 is achieved only because of targeted delivery of active moiety docetaxel which is confirmed by TNF alpha levels reduction and analysis of tumour biopsy homogenate of F-6 group compared to other groups. Docetaxel levels were analyzed and found to be 40-70% higher in F-6 group in comparison to other groups. [Score card after 12 days treatment of drugs in breast cancer 5 (severe), 4 (moderate), 3 (mild), 2 (minimal); 1 (nil).]

The interferon is conjugated most preferably via a terminal reactive group on the polymer although conjugations can also be branched from the non-terminal reactive groups. The polymer with the reactive group(s) is designated herein as “activated polymer”. The reactive group selectively reacts with free amino or other reactive groups on the protein. The activated polymer(s) are reacted so that attachment may occur at any available interferon amino group such as the alpha amino groups or the epsilon-amino groups of lysines. Free carboxylic groups, suitably activated carbonyl groups, hydroxyl, guanidyl, oxidized carbohydrate moieties and mercapto groups of the interferon (if available) can also be used as attachment sites.

The most likely attachment site is determined by the reactive group on the polymer and the reaction conditions.

The activity and stability of the conjugates can be varied in several ways, for example, by using a polymer of different molecular sizes. Solubilities of the conjugates can be varied by changing the proportion and size of the polymer and protein/peptide incorporated in the conjugate, altering carrier of drug moiety and activation procedures of polymer.

An embodiment of the invention is an administration of a therapeutically effective amount of the triple conjugates of the invention to a subject in need thereof who is at risk of developing, for example, one of the diseases described herein above or to a subject already showing such pathologies.

In FIG. 13, an illustration is given for a comparative reduction in TNF alpha levels in tumor induced breast cancer model wherein effect of Docetaxel, Docetaxel plus pegylated interferon and F-6 on Tumour Necrosis Factor α (TNFα) in breast cancer rat model is depicted. It was observed that there is significant increase in (p<0.001) percentage of tumour in breast cancer induced group as compared to control. In case of Docetaxel, docetaxel plus pegylated interferon 2a and F-6, the tumour is significantly inhibited only F-6 treated group on 12^{th} day as compared to 0 day. There was significantly increased tumour volume in breast cancer induced group as compared control group. After treatment with respective drug, the tumour volume was significantly decreased in F-6 along with TNF alpha level in same group.

In a certain aspect, any route of administration compatible with the active drug/principle can be employed. A parenteral administration, such as subcutaneous, intramuscular or intravenous injection is preferred in certain embodiments of the invention.

However, in yet another embodiment, oral or topical or alternate non parenteral routes is adopted. The dose of the active ingredient to be administered depends on the basis of the medical prescriptions according to age, weight and the individual response of the patient.

The formulation of the triple conjugates of the present invention is provided in a pharmaceutically administrable formulation useful for treating the biological conditions or disorders noted herein above to a subject in need thereof according to an embodiment herein.

In preparing the compositions in oral liquid dosage forms including suspensions, elixirs and solutions a typical pharmaceutical media such as water, glycols, oils, alcohols, flavoring agents, preservatives, coloring agents and the like can be employed according to an embodiment herein.

Similarly, when preparing oral solid dosage forms including powders, tablets and capsules, carriers such as starches, sugars, diluents, granulating agents, lubricants, binders, disintegrating agents and the like are employed according to an embodiment herein.

In yet another embodiment, the pharmaceutically administrable formulation for parenteral administration can be prepared in an injectable form comprising the active drug/principle and a suitable vehicle. For parenteral administration, the suitable vehicle may or may not comprise water, although other ingredients that aid in solubility or serve as preservatives may also be included.

Furthermore, an injectable suspensions may also be prepared, in which case an appropriate liquid carrier, suspending agents and the like will be employed. The suitable vehicles for the parenteral administration are well known in the art and include, for example, water, saline solution, Ringer solution and/or dextrose. The suitable vehicle can contain small amounts of excipients in order to maintain the stability and isotonicity of the pharmaceutically administrable formulation. The preparation of the solutions can be carried out according to the ordinary modalities. For topical administration, the present invention may be formulated using bland, moisturizing bases, such as ointments or creams.

The pharmaceutical composition comprising conjugate of the present invention will generally be administered in the form of a dosage unit.

In a preferred embodiment the drug molecule is administered lesser times in said drug(s)-protein-polymer triple conjugate as compared to individual recommended administration of the constituents of the triple conjugate (such as active drug or protein) for the treatment or prevention of a disease owing to sustained release effect.

There have been various challenges and factors that affected a successful and effective formulation employing the novel drug delivery system as described herein above. Some of the critical factors observed include i) Finding a suitable protein/peptide which can target a specific intend site in a cell/tissue/organ(s), ii) Finding a suitable polymer which has compatibility with the protein as well as with a active drug to be carried to the intended target site in the cell/tissue/organ(s), iii) Activation of the polymer to enable multiple conjugation sites opening to accommodate higher amount of active drug, iii) Standardization of conjugation time required for complete conjugation to occur, iv) Controlling and Sustaining the effect of drug by selective polymer binding in to it, v) Standardizing the concentrations of the protein moiety, the polymer moiety and the active drug moiety in conjugate to ensure maximum efficacy with minimum concentration of the active drug used therein.

According to one preferred embodiment, the triple conjugate of drug(s)-protein-polymer for anticancer activity along with immune boosting activity simultaneously with drug targeting and sustained delivery of active moiety is described in herein below. It is observed that for solid tumors specially breast and ovarian tumors, HER2 receptors are over expressed with Tyrosyine kinase. Interferons are found to have selective binding affinity with these receptors. Docetaxel is a preferred molecular entity used for treatment of these indications. In one of the currently available and preferred therapy, a 60-100 mg/m2 of Taxotere (Docetaxel) is administered to a subject in need. However, neutropenia (<2,000 neutrophils/mm3) occurs virtually in all patients who received the above medication and grade 4 neutropenia (<500 cells/mm3) occurs in 85% of patients who received 100 mg/m2 of the above medication and 75% of patients who received n 60 mg/m2 of the same medication as adverse effect.

In the most preferred embodiment, the polymer moiety is selected from a group of polymers preferably PEG having molecular weight <50 KDa, preferably <10 KDa, still preferably <5 KDa, more preferably <2 KDa. The activation of polymer is done by using different activation mechanism known to a person skilled in art including biotinylation. The carrier which is used is selected from a group comprising T-80, PG Ethyl alcohol or other pharmaceutically acceptable solvents and is preferably diethylene glycolmon ethyl ether. The protein moiety includes INFα-2A, INF gamma in an amount from about 0.001 ml to 0.9 ml of 1 to 25 MIU. The protein moiety is mixed with PEG preferably PEG 400 which is pre-activated using NHS biotin, under inter gas atmosphere stirring at a temperature about 1° C. to about 8° C. The solution thus obtained is kept for conjugation for about 0.08 hrs to about to 48 hr under condition of stirring for obtaining the triple conjugate.

Above disclosure describe a manner and method of making and using the invention and sets forth the best mode contemplated by the inventor for carrying out his invention but is not to be construed as limiting. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications and equivalents of the described modes for carrying out the invention that are obvious to those skilled in formulation development or related fields are intended to be within the scope of the invention. The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

1. A pharmaceutical formulation based on novel drug delivery system for targeted delivery of at least one active drug, comprising a triple conjugate, wherein said triple conjugate comprises a therapeutically effective amount of:
i) a protein moiety capable of binding selectively to a particular target site possessed by a cell/organ,
ii) a polymer moiety, covalently linked to said protein moiety;
iii) an active drug moiety comprising of at least one of said active drug and covalently linked to either said protein moiety or to said polymer moiety;
wherein said active drug moiety and said protein moiety are chemically different to each other; and
wherein said active drug moiety is non-biological entity.

2. The pharmaceutical formulation of claim 1,
wherein said protein moiety, said polymer moiety and said active drug moiety are linked to each other.

3. The pharmaceutical formulation of claim 1, and wherein said triple conjugate is administered to a subject in need thereof as a single unit.

4. The pharmaceutical formulation of claim 1,
wherein said polymer moiety is present in an amount ranging from 60% to 85% of the of the formulation;
wherein said protein moiety is present in an amount ranging from 6% to 12% of the formulation; and
wherein said active drug is present in an amount ranging from 10% to 20% of the formulation.

5. The pharmaceutical formulation of claim 1,
wherein molecular weight of said triple conjugate is <50 KDa, preferably <25 Da; and wherein molecular weight of said polymer moiety is >0.2 KDa to <50 KDa,

6. The pharmaceutical formulation of claim 1,
wherein said polymer moiety is selected from the group comprising of polyalkylene glycols, polyethylene glycols (PEG), monomethoxypoly (ethylene glycols), monohydroxypol (ethylene glycols), polyalkylene oxides, polyoxiranes, polyolefinic alcohols,
polycarboxylates, polyvinylpyrrolidones, poly(oxyethyleneoxymethylenes), poly(amino acids), polyacryloylmorpholines, copolymers of amides, alkylene oxides, dextrans, hyaluronic acids, polyacrylamides, carbohydrate-based polymers, polynucleotides, or any combination thereof.

7. The pharmaceutical formulation of claim 1, wherein the formulation further comprises of a carrier,
wherein said carrier is diethyleneglycol monoethyl ether; and
wherein said carrier is present in an amount ranging from 0.01 ml to 0.5 ml.

8. The pharmaceutical formulation of claim 1, wherein the formulation further comprises of a an activation agent,
wherein said activation agent is Sulpho-NHS biotin; and
wherein said activation agent is present in an amount ranging from 0.01 ml to 0.5 ml.

9. The pharmaceutical formulation of claim 1, wherein said active drug is selected from the group comprising of cytotoxic drug, anti-viral drug, anti-neoplastic drug, anti-inflammatory drug, antibiotic, analgesic drug, drug acting on CNS, CVS, proton pump inhibitor, or any combination thereof.

10. The pharmaceutical formulation of claim 1, wherein said active drug moiety is an anticancer drug selected from the group comprising of anti-metabolites masquerade as purines, ciplatin, carboplatin, oxaliplatin, mechlorethamine, cyclophosphamide, chlorambucil, ifosfamide, Vincristine, Vinblastine, Vinorelbine, Vindesine, podophyllotoxins, etoposide teniposide, docetaxel, paclitaxel, irinotecan, topotecan, actinomycin,
anthracyclines, doxorubicin, daunorubicin, valrubicin, idarubicin, epirubicin, bleomycin, plicamycin, mitomycin, dactinomycin, cytarabine, bortezomibe,
fludarabine, clatribine, Gemcitabine, Methotrexate, 5-fluro uracil, Amscrine, Cladribine, Carmustine, or pharmaceutically acceptable salts thereof.

11. The pharmaceutical formulation of claim 1, wherein said protein moiety is selected from the group comprising of immunoglobulins, glycoproteins, antibodies, polypeptides, enzymes, peptides, Interferon (INF), interleukins, hormones, somatomedins, erythropoietin, pigmentary hormones, hypothalamic releasing factors, antidiuretic hormones, prolactin, chorionic gonadotropin, follicle-stimulating hormone, thyroid-stimulating hormone or tissue plasminogen activator.

12. The pharmaceutical formulation of claim 1, wherein said protein moiety is Interferon (INF).

13. The pharmaceutical formulation of claim 12, wherein said protein moiety is selected from the group comprising of INFa-2a, INFa-2b, or INF-γ.

14. The pharmaceutical formulation of claim 12, wherein said INF is present in an amount ranging from 0.001 ml to 1.0 ml.

15. The pharmaceutical formulation of claim 12, wherein said Interferon has 1 to 25 MIU potency.

16. The pharmaceutical formulation of claim 1, wherein said polymer moiety is PEG and is present in an amount ranging from 0.5 ml to 1.0 ml.

17. The pharmaceutical formulation of claim 1, wherein said active drug is docetaxel or a pharmaceutically acceptable salt thereof.

18. The pharmaceutical formulation of claim 17, wherein said active drug is present in an amount ranging from 10 mg to 40 mg.

19. A method of preparation of a pharmaceutical formulation comprising a triple conjugate wherein said triple conjugate comprises a polymer moiety, a protein moiety, and an active drug moiety comprising one or more active drug(s), said method comprising the steps of:
(i) preparing a complex of said polymer moiety and said protein moiety by addition of said polymer moiety to said protein moiety to obtain a polymer-protein complex following addition of said drug moiety; or
(ii) adding said polymer moiety, said protein moiety and said drug moiety simultaneously to facilitate a conjugation;
wherein said active drug moiety and said protein moiety are chemically different to each other; and
wherein said active drug moiety is non-biological entity.

20. The method of preparation of claim 19, wherein said conjugation is facilitated under an inert gas atmosphere and under constant stirring condition at 1° C. to 8° C.

21. The method of preparation of claim 19, wherein said polymer moiety is activated by employing one or more activation agents wherein said activation enables said conjugation of said drug moiety and said protein moiety to said polymer moiety.

22. The method of preparation of claim 21, wherein said activation agent is Sulpho NHS biotin and said protein is Interferon (INF).

23. The method of preparation of claim 19, wherein said polymer moiety is pre-activated.

24. The method of preparation of claim 21, wherein said activation of said polymer occurs when said polymer is added to said activation agent for a period ranging from 0.08 hrs to 24 hrs.

25. The method of preparation of claim 19, wherein said conjugation occurs for a period ranging from 0.5 hrs to 48 hrs.

26. The method of preparation of claim 19, wherein said drug is dissolved in one or more carrier(s) before said drug is added to said polymer moiety or said polymer-protein complex, wherein said carrier is selected from the group comprising of ethylene glycols,
diethyleneglycol mono ethyl ether, diethyleneglycol monoethyl ether, polyalkylene oxides, polyoxiranes, polyolefinic alcohols, polycarboxylates, poly vinylpyrrolidones, poly xyethyleneoxymethylenes, polyamino acids, polyacryloylmorpholines, copolymers of amides, alkylene oxides, dextrans, hyaluronic acids, or polyacrylamides.

27. The method of preparation as claimed in claim 19, wherein said drug is docetaxel or a pharmaceutically acceptable salt thereof.

28. A method of treating or preventing a disease condition selected form the group comprising of neoplastic diseases, autoimmune diseases, GERD, Ulcer, Autoimmune conditions, Diabetes, Genetic conditions, Viral/Bacterial/Parasitic Infections, Worm conditions, Physical conditions, Prion diseases, Nutritional deficiencies, Vitamin/Mineral deficiencies Mitochondrial diseases, Accidents, Sexually Transmitted Diseases, Pregnancy Conditions, Breastfeeding Conditions, Birth defects, Male/Female/Infant/childhood/Adolescent conditions, Immune disorders, Balance disorders, Pain, Systemic disorders, Blood conditions, Blood vessel conditions, Nerve conditions, Muscle conditions, Heart conditions, Back/Neck/Spinalcord conditions, Eye conditions, Brain conditions, Mental conditions, Nose conditions, Mouth conditions, Dental conditions, Foot/Leg/Knee conditions, upper limb condition, Shoulder conditions, Ear conditions, Lung conditions, Liver conditions, Kidney conditions, Gall bladder conditions, Pancreas conditions, Digestive conditions, Prostate conditions, Male genital conditions, Obstetrical conditions, Gynaecological conditions, Thyroid disorders, or Hearing disorders, comprising administering a therapeutically effective amount of pharmaceutical formulation comprising triple conjugate wherein said triple conjugate comprises a polymer moiety, a protein moiety and an active drug moiety, to a subject in need thereof;
wherein said active drug moiety and said protein moiety are chemically different to each other; and
wherein said active drug moiety is non-biological entity.

29. The method of 28, wherein said pharmaceutical formulation is administered through a parenteral route.

30. The method of claim 28, wherein said drug is a cytotoxic drug.

31. The method of claim 28, wherein said drug is docetaxel or a pharmaceutically acceptable salt thereof.

32. The method of claim 24, wherein amount of said drug administered is from 10 mg to 40 mg.

33. (canceled)
